(19) United States
(12) Patent Application Publication
(10) Pub. No.: US 2002/0169107 A1
(43) Pub. Date: Nov. 14, 2002

(54) Novel aromatic azides for type I phototherapy

(75) Inventors: Raghavan Rajagopalan, Beechwood, OH (US); Gary Cantrell, Troy, IL (US); Samuel I. Achilefu, St. Louis, MO (US); Joseph E. Bugaj, St. Charles, MO (US); Richard B. Dorshow, St. Louis, MO (US)

Correspondence Address: David E. Jefferies, WOOD, HERRON & EVANS, L.L.P., 2700 Carew Tower, 441 Vine Street, Cincinnati, OH, 45202-2917, US

(73) Assignee: Mallinckrodt Inc.

(21) Appl. No.: 09/766,347

(22) Filed: Jan. 19, 2001

(51) Int. Cl. A61K 38/16; A61K 31/70; A61K 31/665; C07J 41/00; C07K 16/00; C07K 14/00

(52) U.S. Cl. 514/2; 514/54; 514/151; 530/409; 536/18.7; 552/1; 552/9

(57) ABSTRACT

The present invention discloses novel aromatic azide derivatives and their bioconjugates for phototherapy of tumors and other lesions. The organic azides of the present invention are designed to absorb low-energy ultraviolet, visible, or near-infrared (NIR) region of the electromagnetic spectrum. The phototherapeutic effect is caused by direct interaction of nitrene, the reactive intermediate produced upon photoexcitation of the aromatic azide, with the tissue of interest. The compounds of the present invention are administered to a patient, allowed to accumulate at the site of the tumor or other lesion, and are exposed to light in order to perform a phototherapeutic procedure.

### FIELD OF THE INVENTION

The present invention relates to phototherapy using novel organic azide compounds.

### BACKGROUND OF THE INVENTION

The use of visible and near-infrared (NIR) light in clinical practice is growing rapidly. Compounds absorbing or emitting light in the visible or near infrared (NIR), or long-wavelength (UV-A,>350 nm) region of the electromagnetic spectrum are potentially useful for optical tomographic imaging, endoscopic visualization, and phototherapy. However, a major advantage of biomedical optics lies in its therapeutic potential. Phototherapy has been demonstrated to be a safe and effective procedure for the treatment of various surface lesions, both external and internal. Its efficacy is akin to radiotherapy, but without the harmful radiotoxicity to critical non-target organs.

Phototherapy has been in existence for many centuries and has been used to treat various skin surface ailments. As early as 1400 B.C. in India, plant extracts (psoralens), in combination with sunlight, were used to treat vitiligo. In 1903, Von Tappeiner and Jesionek, used eosin as a photosensitizer for treating skin cancer, lupus of the skin, and condylomata of female genitalia. Over the years, the combination of psoralens and ultraviolet A (low-energy) radiation has been used to treat a wide variety of dermatological diseases including psoriasis, parapsoriasis, cutaneous T-cell lymphoma, eczema, vitiligo, areata, and neonatal bilirubinemia. Although the potential of cancer phototherapy has been recognized since the early 1900's, systematic studies to demonstrate safety and efficacy began only in 1967 with the treatment of breast carcinoma. In 1975, Dougherty et al. conclusively established that long-term cure is possible with photodynamic therapy (PDT). Currently, phototherapeutic methods are also being investigated for the treatment of some cardiovascular disorders such as atherosclerosis and vascular restenosis, for the treatment of rheumatoid arthritis, and for the treatment of some inflammatory diseases such as Chron's disease.

Phototherapeutic procedures require photosensitizers (i.e. chromophores) having high absorptivity. These compounds should preferably be chemically inert, and become activated only upon irradiation with light of an appropriate wavelength. Selective tissue injury can be induced with light when photosensitizers bind to the target tissues, either directly or through attachment to a bioactive carrier. Furthermore, if the photosensitizer is also a chemotherapeutic agent (e.g., anthracycline antitumor agents), then an enhanced therapeutic effect can be attained.

Effective phototherapeutic agents require the following: (a) large molar extinction coefficients, (b) long triplet lifetimes, (c) high yields of singlet oxygen and/or other reactive intermediates, viz., free radicals, nitrenes, carbenes, or open-shell ionic species such as cabonium ions and the like, (d) efficient energy or electron transfer to cellular components, (e) low tendency for aggregation in an aqueous milieu, (f) efficient and selective targeting of lesions, (g) rapid clearance from the blood and non-target tissues, (h) low systemic toxicity, and (i) lack of mutagenicity.

Photosensitizers operate via two distinct mechanisms, termed Types 1 and 2. Type 1 mechanisms are shown in the following scheme:

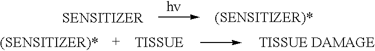

Type 1 mechanisms involve direct energy or electron transfer from the photosensitizer to the cellular components thereby causing cell death. Type 2 mechanisms involve two distinct steps, as shown in the following scheme:

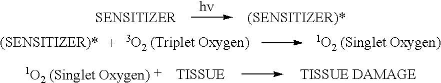

In the first step, singlet oxygen is generated by energy transfer from the triplet excited state of the photosensitizer to the oxygen molecules surrounding the tissues. In the second step, collision of singlet oxygen with the tissues promotes tissue damage. In both Type 1 and Type 2 mechanisms, the photoreaction proceeds via the lowest triplet state of the sensitizer. Hence, a relatively long triplet lifetime is required for effective phototherapy, whereas a relatively short triplet lifetime is required to avoid photodamage for photodiagnostics.

The biological basis of tissue injury brought about by tumor phototherapeutic agents has been the subject of intensive study. Various biochemical mechanisms for this tissue injury have been postulated based on the very limited number of photosensitizers studied. These biochemical mechanisms are as follows: a) cancer cells upregulate the expression of low density lipoprotein (LDL) receptors, and photodynamic therapy (PDT) agents bind to LDL and albumin selectively; (b) porphyrin-like substances are selectively taken up by proliferative neovasculature; (c) tumors often contain increased number of lipid bodies and are thus able to bind to hydrophobic photosensitizers; (d) a combination of ‘leaky’ tumor vasculature and reduced lymphatic drainage causes porphyrin accumulation; (e) tumor cells may have increased capabilities for phagocytosis or pinocytosis of porphyrin aggregates; (f) tumor associated macrophages may be largely responsible for the concentration of photosensitizers in tumors; and (g) cancer cells may undergo apoptosis induced by photosensitizers. Among these mechanisms, (f) and (g) are the most general and, of these two alternatives, there is a general consensus that (f) is the most likely mechanism by which the phototherapeutic effect of porphyrin-like compounds is induced.

Most of the currently known photosensitizers are commonly referred to as ‘photodynamic therapy (PDT)’ agents and operate via the Type 2 mechanism. For example, Photofrin II (a hematoporphyrin derivative) has been recently approved by the United States Food and Drug Administration for the treatment of bladder, esophageal, and late-stage lung cancers. However, Photofrin II has been shown to have several drawbacks: a low molar absorptivity (ε=3000 M⁻¹), a low singlet oxygen quantum yield (φ=0.1), chemical heterogeneity, aggregation, and prolonged cutaneous photosensitivity. Hence, there has been considerable effort in developing safer and more effective photosensitizers for PDT which exhibit improved light absorbance properties, better clearance, and decreased skin photosensitivity compared to Photofrin II. These include monomeric porphyrin derivatives, corrins, cyanines, phthalocyanines, phenothiazines, rhodamines, hypocrellins, and the like. However, these phototherapeutic agents mainly operate via the Type 2 mechanism.

Surprisingly, there has not been much attention directed at developing Type 1 phototherapeutic agents, despite the fact that the Type 1 mechanism appears to be inherently more efficient than the Type 2 mechanism. First, unlike Type 2, Type 1 photosensitizers do not require oxygen for causing cellular injury. Second, the Type 1 mechanism involves two steps (photoexcitation and direct energy transfer), whereas the Type 2 mechanism involves three steps (photoexcitation, singlet oxygen generation, and energy transfer). Furthermore, certain tumors have hypoxic regions, which renders the Type 2 mechanism ineffective. However, in spite of the drawbacks associated with the Type 2 mechanism, only a small number of compounds have been developed that operate through the Type 1 mechanism, e.g. anthracylines antitumor agents.

Thus, there is a need to develop effective phototherapeutic agents that operate via the Type 1 mechanism. Phototherapeutic efficacy can be further enhanced if the excited state photosensitizers can generate reactive intermediates such as free radicals, nitrenes, carbenes, and the like, which have much longer lifetimes than the excited chromophore and have been shown to cause considerable cell injury.

### SUMMARY

The present invention discloses novel, organic azide derivatives and their bioconjugates for phototherapy of tumors and other lesions. More specifically, the present invention discloses organic azide compounds having the formula:

E—L—Ar—X—N₃

N₃ is the azide moiety that produces nitrene upon photoactivation. Ar is a chromophore that undergoes sensitization. This chromophore (Ar) is an aromatic or a heteroaromatic radical derived from the group consisting of benzenes, polyfluorobenzenes, naphthalenes, naphthoquinones, anthracenes, anthraquinones, phenanthrenes, tetracenes, naphthacenediones, pyridines, quinolines, isoquinolines, indoles, isoindoles, pyrroles, imidiazoles, pyrazoles, pyrazines, purines, benzimidazoles, benzofurans, dibenzofurans, carbazoles, acridines, acridones, phenanthridines, thiophenes, benzothiophenes, dibenzothiophenes, xanthenes, xanthones, flavones, coumarins, and anthacylines. E is an epitope and is selected from the group consisting of somatostatin receptor binding molecules, ST receptor binding molecules, neurotensin receptor binding molecules, bombesin receptor binding molecules, CCK receptor binding molecules, steroid receptor binding molecules, and carbohydrate receptor binding molecules. L is a linker between the chromophore and the epitope and is selected from the group consisting of —(CH₂)ₐ—, —(CH₂)_{b}CONR¹—, —N(R²)CO(CH₂)_{c}—, —OCO(CH₂)_{d}—, —(CH₂)ₑCO₂—, —OCONH—, —OCO₂—, —HNCONH—, —HNCSNH—, —HNNHCO—, —OSO₂—, —NR³(CH₂)ₑCONR⁴—, —CONR⁵(CH₂)_{f} NR⁶CO—, and —NR⁷CO(CH₂)_{g}CONR⁸—. X is either a single bond or is selected from the group consisting of —(CH₂)ₕ—, —OCO—, —HNCO—, —(CH₂)ᵢCO—, and —(CH₂)ⱼOCO—. R¹ to R⁸ are independently selected from the group consisting of hydrogen, C1-C10 alkyl, —OH, C1-C10 polyhydroxyalkyl, C1-C10 alkoxyl, C1-C10 alkoxyalkyl, —SO₃H, —(CH₂)ₖCO₂H, and —(CH₂)ₗNR⁹R¹⁰. R⁹ and R¹⁰ are independently selected from the group consisting of hydrogen, C1-C10 alkyl, C5-C10 aryl, and C1-C10 polyhydroxyalkyl. And a to l independently range from 0 to 10.

The present invention also discloses a method of performing a phototherapeutic procedure using the organic azide compounds of the present invention. This method includes the following steps. First, an effective amount of an organic azide photosensitizer having the formula

E—L—Ar—X—N₃

is administered to a subject. Ar is an aromatic or a heteroaromatic radical derived from the group consisting of benzenes, polyfluorobenzenes, naphthalenes, naphthoquinones, anthracenes, anthraquinones, phenanthrenes, tetracenes, naphthacenediones, pyridines, quinolines, isoquinolines, indoles, isoindoles, pyrroles, imidiazoles, pyrazoles, pyrazines, purines, benzimidazoles, benzofurans, dibenzofurans, carbazoles, acridines, acridones, phenanthridines, thiophenes, benzothiophenes, dibenzothiophenes, xanthenes, xanthones, flavones, coumarins, and anthacylines; E is a hydrogen atom or is selected from the group consisting of somatostatin receptor binding molecules, ST receptor binding molecules, neurotensin receptor binding molecules, bombesin receptor binding molecules, CCK receptor binding molecules, steroid receptor binding molecules, and carbohydrate receptor binding molecules; L is selected from the group consisting of —(CH₂)ₐ—, —(CH₂)_{b}CONR¹—, —N(R²)CO(CH₂)_{c}—,—OCO(CH₂)_{d}—, —(CH₂)ₑCO₂—, —OCONH—, —OCO₂—, —HNCONH—, —HNCSNH—, —HNNHCO—, —OSO₂—, —NR³(CH₂)ₑCONR⁴—, —CONR⁵(CH₂)_{f}NR⁶CO—, and —NR⁷CO(CH₂)_{g}CONR⁸—; X is either a single bond or is selected from the group consisting of —(CH₂)ₕ—, —OCO—, —HNCO—, —(CH₂)ᵢCO—, and —(CH₂)ⱼOCO—; R¹ to R⁸ are independently selected from the group consisting of hydrogen, C1-C10 alkyl, —OH, C1-C10 polyhydroxyalkyl, C1-C10 alkoxyl, C1-C10 alkoxyalkyl, —SO₃H, —(CH₂)ₖCO₂H, and —(CH₂)ₗNR⁹R¹⁰; R⁹ and R¹⁰ are independently selected from the group consisting of hydrogen, C1-C10 alkyl, C5-C10 aryl, and C1-C10 polyhydroxyalkyl; and a to I independently range from 0 to 10. Second, the photosensitizer is allowed to accumulate in target tissue. And finally, the target tissues are exposed to light of wavelength between 300 and 950 nm with sufficient power and fluence rate to perform the procedure.

In the process outlined above, the photoexcitation of the aromatic chromophore effects a rapid intramolecular energy transfer to the azido group, resulting in bond rupture and production of nitrene and nitrogen gas. The nitrogen that is released is in a vibrationally excited state, which may cause additional cellular injury.

For targeting purposes, external attachment of an epitope is used. If the aromatic azido compounds themselves preferentially accumulate in the target tissue, however, an additional binding group may not be needed. For example, if Ar is an anthracycline moiety, it will bind to cancer cells directly and not require an epitope for targeting purposes.

These and other advantages and embodiments of the inventive compounds and methods will be apparent in light of the following figures, description, and examples.

### BRIEF DESCRIPTION OF THE DRAWINGS

FIG. 1 is a schematic pathway for activation of the inventive compounds.

FIG. 2 is a schematic pathway for the synthesis of a tetrafluorophenylazide derivative.

FIG. 3 is a schematic pathway for the synthesis of an acridone derivative.

FIG. 4 is a schematic pathway for the synthesis of an azidoxanthone derivative.

FIG. 5 is a schematic pathway for the synthesis of an azidoanthraquinone derivative.

FIG. 6 is a schematic pathway for the synthesis of an azidophenanthridene derivative.

FIG. 7 is a schematic pathway for a steroid-photosensitizer conjugate.

FIG. 8 is a schematic pathway for a photosensitizer attached to a biosynthetic intermediate.

### DETAILED DESCRIPTION

The invention discloses novel organic azide derivatives and their bioconjugates for phototherapy of tumors and other lesions. The compounds have the general formula:

E—L—Ar—X—N₃

wherein Ar is an aromatic or a heteroaromatic radical derived from the group consisting of benzenes, polyfluorobenzenes, naphthalenes, naphthoquinones, anthracenes, anthraquinones, phenanthrenes, tetracenes, naphthacenediones, pyridines, quinolines, isoquinolines, indoles, isoindoles, pyrroles, imidiazoles, pyrazoles, pyrazines, purines, benzimidazoles, benzofurans, dibenzofurans, carbazoles, acridines, acridones, phenanthridines, thiophenes, benzothiophenes, dibenzothiophenes, xanthenes, xanthones, flavones, coumarins, and anthacylines; E is either a hydrogen atom or is selected from the group comprising antibodies, peptides, peptidomimetics, carbohydrates, glycomimetics, drugs, hormones, or nucleic acids; L is a linker unit selected from the group comprising —(CH₂)ₐ—, —(CH₂)_{b}CONR¹—, —N(R²)CO(CH₂)_{c}—, —OCO(CH₂)_{d}—, —(CH₂)ₑCO₂—, —OCONH—, —OCO₂—, —HNCONH—, —HNCSNH—, —HNNHCO—, —OSO₂—, —NR³(CH₂)ₑCONR⁴—, —CONR⁵(CH₂)_{f}NR⁶CO—, and —NR⁷CO(CH₂)_{g}CONR⁸—; X is either a single bond or is selected from the group consisting of —(CH₂)ₕ—, —CO—, —OCO—, —HNCO—, —(CH₂)ᵢCO—, and —(CH₂)ⱼOCO—; R¹ to R⁸ are independently selected from the group consisting of hydrogen, C₁-C10 alkyl, —OH, C1-C10 polyhydroxyalkyl, C1-C10 alkoxyl, C1-C10 alkoxyalkyl, —SO₃H, —(CH₂)ₖCO₂H, or —(CH₂)ₗNR⁹R¹⁰; R⁹ and R¹⁰ are independently selected from the group consisting of hydrogen, C1-C10 alkyl, C5-C10 aryl, or C1-C10 polyhydroxyalkyl; and a to I independently range from 0 to 10.

In a first embodiment, azides according to the present invention have the general formula shown above wherein Ar is an aromatic radical derived from the group consisting of benzenes, polyfluorobenzenes, anthracenes, anthraquinones, naphthacenediones, quinolines, isoquinolines, indoles, acridines, acridones, phenanthridines, xanthenes, xanthones, and anthacylines; E is selected from the group consisting of somatostatin receptor binding molecules, ST receptor binding molecules, neurotensin receptor binding molecules, bombesin receptor binding molecules, cholecystekinin receptor binding molecule, steroid receptor binding molecules, and carbohydrate receptor binding molecules; L is selected from the group consisting of —HNCO—, —CONR¹—, —HNCONH—, —HNCSNH—, —HNNHCO—,—(CH₂)ₐCONR¹—, —CONR¹ (CH₂)ₐNR²CO—, and —NR¹CO(CH₂)ₐCONR²—; R¹ and R² are independently selected from the group consisting of hydrogen, C1-C10 alkyl, C1-C10 polyhydroxyalkyl; and a, b, and c independently range from 0 to 6.

In a second embodiment, azides according to the present invention have the general formula shown above wherein Ar is selected from the group consisting of tetrafluorobenzenes, phenanthridines, xanthones, anthraquinones, acridines, and acridones; E is a selected from the group consisting of octreotide and octreotate peptides, heat-sensitive bacterioendotoxin receptor binding peptides, carcinoembryonic antigen antibody (anti-CEA), bombesin receptor binding peptide, neurotensin receptor binding peptide, cholecystekinin receptor binding peptide, and estrogen steroids; L is selected from the group consisting of —HNCO—, —CONR¹—, —HNCSNH—, —HNNHCO—, —(CH₂)ₐCONR¹—,—CONR¹(CH₂)ₐNR²CO—; and R¹ and R² are independently selected from the group consisting of hydrogen, C1-C10 alkyl, C1-C5 polyhydroxyalkyl; and a, b, and c independently range from 0 to 6.

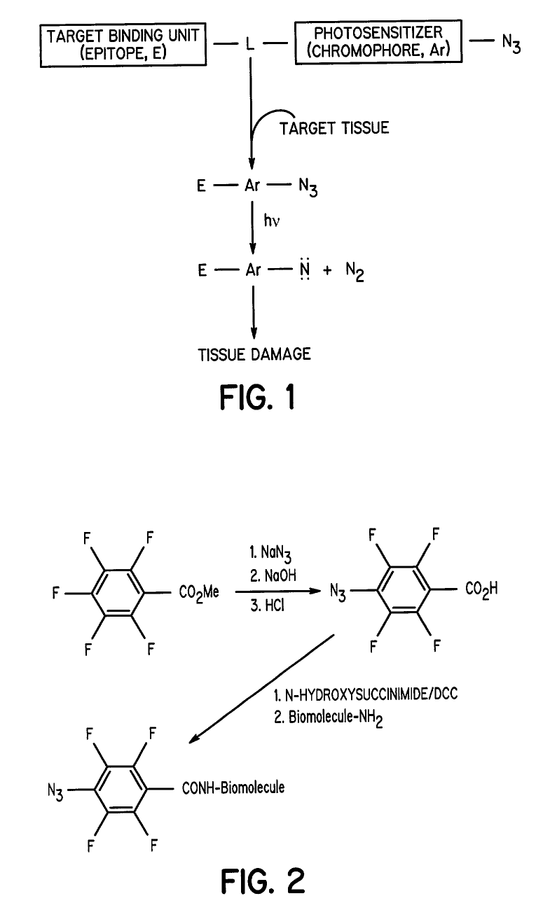

These compounds operate mainly by a Type I mechanism as shown in FIG. 1. In the compounds according to the present invention, N₃ is the azide moiety that produces nitrene upon photoactivation, and Ar is an aromatic chromophore that undergoes photosensitization. Aliphatic azido compounds can also be used for phototherapy, but may require high-energy light for activation unless the azide moiety is attached to conjugated polyene system. L is a linker between the chromophore and the epitope. Epitope (E) is a particular region of the molecule that is recognized by, and binds to, the target surface. An epitope is usually, but not always, associated with biomolecules which include hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Specific examples of biomolecules include steroid hormones for the treatment of breast and prostate lesions, somatostatin, bombesin, and neurotensin receptor binding molecules for the treatment of neuroendocrine tumors, cholecystekinin receptor binding molecules for the treatment of lung cancer, heat sensitive bacterioendotoxin (ST) receptor and carcinoembryonic antigen (CEA) binding molecules for the treatment of colorectal cancer, dihyroxyindolecarboxylic acid and other melanin producing biosynthetic intermediates for melanoma, integrin receptor and atheroscleratic plaque binding molecules for the treatment of vascular diseases, and amyloid plaque binding molecules for the treatment of brain lesions. Examples of synthetic polymers include polyaminoacids, polyols, polyamines, polyacids, oligonucleotides, aborols, dendrimers, and aptamers.

Coupling of diagnostic and radiotherapeutic agents to biomolecules can be accomplished by methods well known in the art, as disclosed in Hnatowich et al., Radiolabeling of Antibodies: A simple and efficient method. Science, 1983, 220, 613; A. Pelegrin et al., Photoimmunodiagnostics with antibody-fluorescein conjugates: in vitro and in vivo preclinical studies. Journal of Cellular Pharmacology, 1992, 3, 141-145, and U.S. Pat. No. 5,714,342, which are expressly incorporated by reference herein in their entirety. Successful specific targeting of fluorescent dyes to tumors using antibodies and peptides for diagnostic imaging of tumors has been demonstrated by us and others, for example, S. A. Achilefu et al., Novel receptor-targeted fluorescent contrast agents for in vivo imaging of tumors. Investigative Radiology, 2000, 35(8), 479-485; B. Ballou et al., Tumor labeling in vivo using cyanine-conjugated monoclonal antibodies. Cancer Immunology and Immunotherapy, 1995, 41, 257-263; K. Licha et al., New contrast agent for optical imaging: acid-cleavable conjugates of cyanine dyes with biomolecules. In Biomedical Imaging: Reporters, Dyes, and Instrumentation, D. J. Bornhop, C. Contag, and E. M. Sevick-Muraca (Eds.), Proceedings of SPIE, 1999, 3600, 29-35, which are expressly incorporated by reference herein in their entirety. Therefore, the inventive receptor-targeted phototherapeutic agents are expected to be effective in the treatment of various lesions.

In the process outlined in FIG. 1, the photoexcitation of the aromatic chromophore effects rapid intramolecular energy transfer to the azido group, resulting in bond rupture and production of nitrene and nitrogen gas. The nitrogen that is released is in a vibrationally excited state, which may cause additional cellular injury.

For targeting purposes, external attachment of an epitope is used. If the aromatic azido compounds themselves preferentially accumulate in the target tissue, however, an additional binding group may not be needed. For example, if Ar is an anthracycline moiety, it will bind to cancer cells directly and not require an epitope for targeting purposes.

The synthesis of azido compounds is accomplished by a variety of methods known in the art, such as disclosed in S. R. Sandler and W. Karo, Azides. In Organic Functional Group Preparations (Second Edition), pp. 323-349, Academic Press: New York, 1986, which is expressly incorporated by reference herein in its entirety. Aromatic azides derived from acridone, xanthone, anthraquinone, phenanthridine, and tetrafluorophenyl systems have been shown to photolyze in the visible and in UV-A regions, for example, L. K. Dyall and J. A. Ferguson, Pyrolysis of aryl azides. XI Enhanced neighbouring group effects of carbonyl in a locked conformation. Australian Journal of Chemistry, 1992, 45, 1991-2002; A. Y. Kolendo, Unusual product in the photolysate of 2-azidoxanithone. Chemistry of Heterocyclic Compounds, 1998, 34(10), 1216; R. Theiler, Effect of infrared and visible light on 2-azidoanthraquinone in the QA binding site of photosynthetic reaction centers. An unusual mode of activation of photoaffinity label. Biological Chemistry Hoppe-Seyler, 1986, 367(12), 1197-207; C. E. Cantrell and K. L. Yielding, Repair synthesis in human lymphocytes provoked by photolysis of ethidium azide. Photochemistry and Photobiology, 1977, 25(2), 1889-191; and R. S. Pandurangi et al., Chemistry of bifunctional photoprobes 3: correlation between the efficiency of CH insertion by photolabile chelating agents. First example of photochemical attachment of 99mTc complex with human serum albumin. Journal of Organic Chemistry, 1998, 63, 9019-9030, each of which is expressly incorporated by reference herein in its entirety. The inventive azide derivatives contain additional functionalities that can be used to attach various types of biomolecules, synthetic polymers, and organized aggregates for selective delivery to various organs or tissues of interest. Preparations of representative compounds from the preferred embodiment are outlined in FIGS. 2-5.

A typical preparation of a tetrafluorophenylazide derivative is shown in FIG. 2. Methyl 2,3,4,5,6-pentafluorophenylbenzoate is reacted with sodium azide in aqueous acetone, and the resulting azidoester is saponified with sodium hydroxide to give 4-azido-2,3,5,6-tetrafluorobenzoic acid. The azidoacid is then converted to the corresponding active ester using N-hydroxysuccimide (NHS) and dicyclohexylcarbodiimide (DCC). The active ester can be attached to any desired biomolecule of interest. Specifically, the biomolecules bind to colorectal, cervical, ovarian, lung, and neuroendocrine tumors, and include somatostatin, cholesystekinin, bombesin, neuroendrocrine, and ST receptor binding compounds.

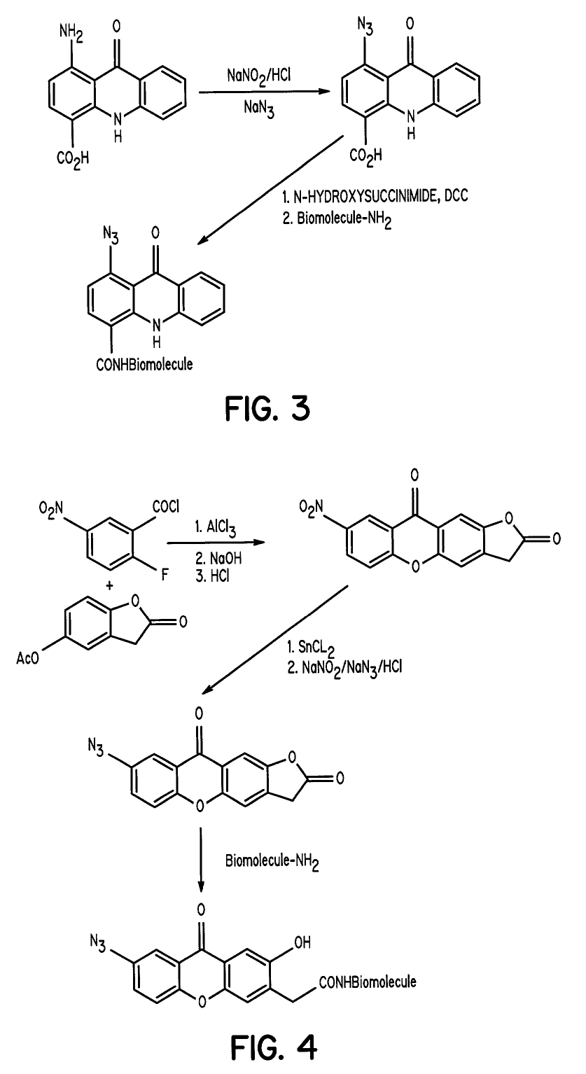

An acridone derivative is prepared according to FIG. 3. The starting aminoacridone is converted to the azide by a standard method of diazotization of the amino group and displacement of the diazonium group with sodium azide, as disclosed in K. Matsumura, 1-Aminoacridine-4-carboxylic acid. Journal of the American Chemical Society, 1938, 32, 591-592, which is expressly incorporated by reference herein in its entirety. The azide is then conjugated to the biomolecules directly using an automated peptide synthesizer, or indirectly by the active ester route.

A typical preparation of an azidoxanthone derivative is outlined in FIG. 4. The acid chloride is reacted with the lactone under Friedel-Crafts conditions to give the benzophenone intermediate, which is saponified and cyclized at once to the nitroxanthone. The nitro group is then converted to the azide by a standard sequence of reactions, that is, reduction, diazotization, and sodium azide treatment. The lactone ring should be sufficiently reactive for conjugation to biomolecules mentioned previously.

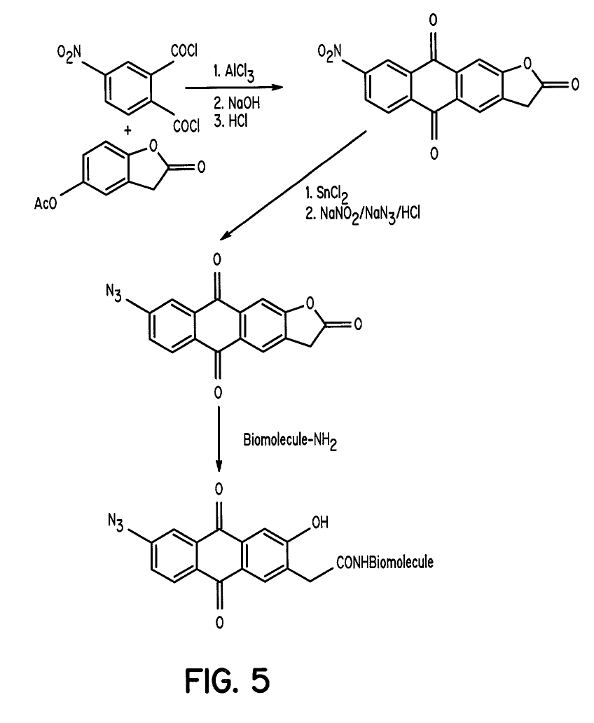

Azidoanthraquinone derivatives can be synthesized according to FIG. 5. The diacid chloride is reacted with the lactone under Friedel-Crafts conditions to the corresponding nitroanthraquinone. The nitrogroup is then converted to the azido group by the standard procedure previously described. The lactone ring is sufficiently reactive for conjugation to the desired biomolecule or, alternatively, it could be hydrolyzed to the acid and then coupled to the biomolecule by conventional methods.

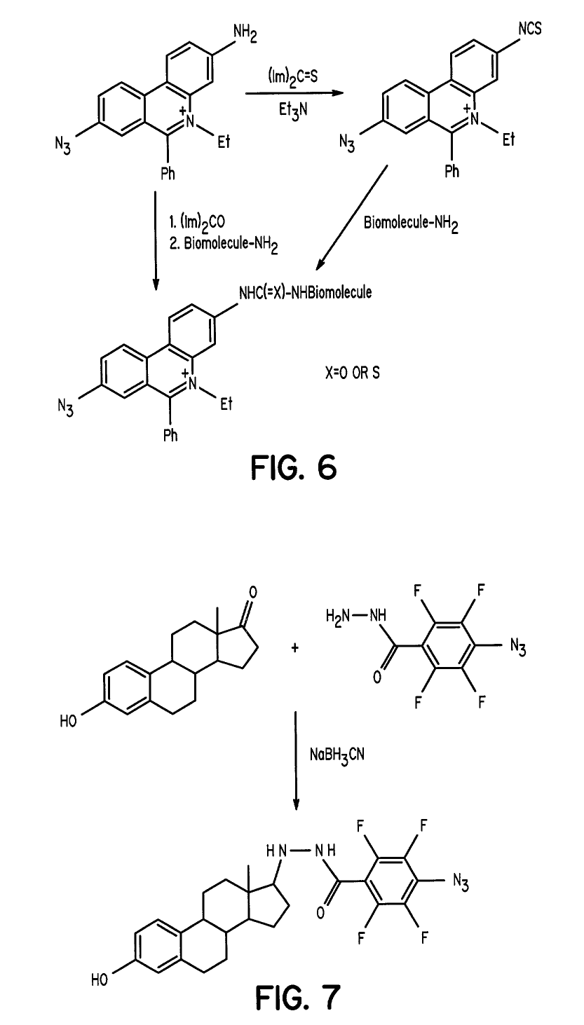

The azidophenanthridene derivatives can be prepared according to FIG. 6. Preparation of the starting material, ethidium azide, has been described in C. E. Cantrell and K. L. Yielding, Binding of ethidium monoazide to the chromatin in human lymphocytes. Biochimica and Biophyica Acta, 1980, 609(1), 173-179, which is expressly incorporated by reference herein in its entirety. The amino group can be activated in several ways. In particular, it can be converted to an isothiocyanate derivative using thiocarbonyl diimidazole or thiophosgene, or it can also be directly condensed with a biomolecule using disuccinimidyl carbonate or carbonyl diimidazole.

The novel compositions of the present invention may vary widely depending on the contemplated application. For tumors, the biomolecule is selected from the class of tumor markers including, but not limited to, somatostatin, bombesin, neurotensin, cholesytekinin, ST, estrogen, and progesterone receptor binding compounds. For vascular lesions, the biomolecule may be selected from the class of integirins, selecting, vascular endothelial growth factor, fibrins, tissue plasminogen activator, thrombin, LDL, HDL, Sialyl Lewis^{X} and its mimics, and atherosclerotic plaque binding compounds. A typical synthetic scheme of a steroid-photosensitizer conjugate is shown in FIG. 7.

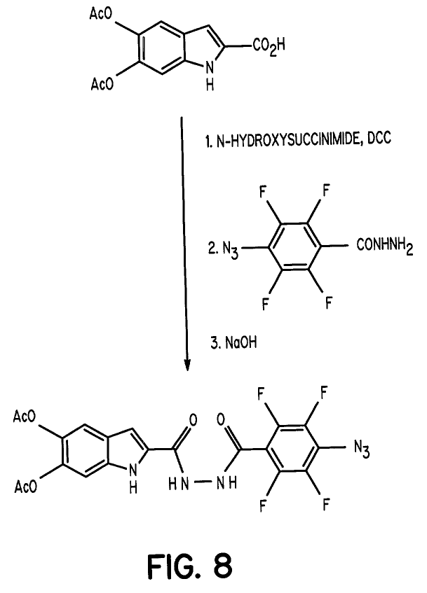

As previously discussed, some compounds accumulate in tumors or other lesions without the assistance of a bioactive carrier. Administration of δ-aminolevulinic acid, an intermediate in porphyrin biosynthesis, results in a two-fold uptake of porphyrins in tumors compared to normal tissues. Similarly, administration of dihydroxyindole-2-carboxylic acid, an intermediate in melanin biosynthesis, produces substantially enhanced levels of melanin in melanoma cells compared to normal cells. Thus, a photosensitizer may be delivered to the site of lesion by attaching it to a biosynthetic intermediate, as shown in FIG. 8.

Methods of performing therapeutic procedures with the inventive compositions are also disclosed. An effective amount of the inventive composition in a pharmaceutically acceptable formulation is administered to a patient. The dose of the photosensitizer may vary from 0.1 to 500 mg/kg body weight, preferably from 0.5 to 2 mg/kg body weight. The photosensitizer is allowed to accumulate in the region of interest, followed by illumination with the light of wavelength 300 to 1200 nm, preferably 350 to 850 nm, at the site of the lesion. If the lesion is on the skin surface, the photosensitizer can be directly illuminated; otherwise, endoscopic catheters equipped with a light source may be employed to achieve phototherapeutic effect. The intensity, power, duration of illumination, and the wavelength of the light may vary widely depending on the location and site of the lesions. The fluence rate is preferably, but not always, kept below 200 mW/cm² to minimize thermal effects. Appropriate power depends on the size, depth, and the pathology of the lesion. The inventive compositions have broad clinical utility which includes, but is not limited to, phototherapy of tumors, inflammatory processes, and impaired vasculature.

The inventive compositions can be formulated into diagnostic or therapeutic compositions for enteral, parenteral, topical, cutaneous, oral, or rectal administration. Topical or cutaneous delivery of the photosensitizer may also include aerosol formulation. The compositions are administered in doses effective to achieve the desired diagnostic: or therapeutic objective. Such doses may vary widely depending upon the particular complex employed, the organs or tissues to be examined, the equipment employed in the clinical procedure, and the like. These compositions contain an effective amount of the phototherapeutic agent, along with conventional pharmaceutical carriers and excipients appropriate for the type of administration contemplated. These compositions may also include stabilizing agents and skin penetration enhancing agents. For example, parenteral administration advantageously contains a sterile aqueous solution or suspension of the photosensitizer in a concentration ranging from about 1 nM to about 0.5 M. Preferred parenteral formulations have a concentration of 1 μM to 10 mM photosensitizer. Such solutions also may contain pharmaceutically acceptable buffers, emulsifiers, surfactants, and, optionally, electrolytes such as sodium chloride. Formulations for enteral administration may vary widely, as is well known in the art. In general, such formulations are liquids, which include an effective amount of the complexes in aqueous solution or suspension. Such enteral composition may optionally include buffers, surfactants, emulsifiers, thixotropic agents, and the like. Compositions for oral administration may also contain flavoring agents and other ingredients for enhancing their organoleptic qualities. Formulations for topical delivery may also contain liquid or semisolid excipients to assist in the penetration of the photosensitizer. The compositions may also be delivered in an aerosol spray.

The following example illustrates a specific embodiment of the invention pertaining to the preparation and properties of a typical bioconjugate derived from bombesin, a bioactive peptide, and a phototherapeutic molecule, 4-azido-2,3,5,6-tetrafluorophenylbenzoic acid.

### EXAMPLE

### Synthesis of 4-azido-2,3,5,6-tetrafluorophenylbenzoate-bombesin (7-14) conjugate

The peptide was prepared by fluorenylmethoxycarbonyl (Fmoc) solid phase peptide synthesis strategy with a commercial peptide synthesizer from Applied Biosystems (Model 432A SYNERGY Peptide Synthesizer). The first peptide cartridge contained Wang resin pre-loaded with an amide resin on 25-μmole scale. The amino acid cartridges were placed on the peptide synthesizer and the product was synthesized from the C- to the N-terminal position. Coupling of the Fmoc-protected amino acids (75 μmol) to the resin-bound free terminal amine (25 μmol) was carried out with 2-(1H-benzotriazol-1-yl)-1,1,3,3-tetramethyluronium hexafluorophosphate (HBTU, 75 μmol)/N-hydroxybenzotriazole (HOBt, 75 μmol). Each Fmoc protecting group on solid support was removed with 20% piperidine in dimethylformamide before the subsequent amino acid was coupled to it. The last cartridge contained 4-azido-2,3,5,6-tetrafluorobenzoic acid, which was successfully coupled to the peptide automatically, thus avoiding the need for post-synthetic manipulations.

After the synthesis was completed, the product was cleaved from the solid support with a cleavage mixture containing trifluoroacetic acid (85%):water (5%):phenol (5%):thioanisole (5%) for 6 hours. The peptide-azide conjugate was precipitated with t-butyl methyl ether and lyophilized in water:acetonitrile (2:3) mixture. The conjugate was purified by HPLC and analyzed with LC/MS, which indicated that the desired compound was obtained in 99% HPLC purity. The azido-bombesin (7-14) conjugate has the following molecular structure: p-azidotetrafluorobenzoyl-Gln—Trp—Ala—Val—Gly—His—Leu—Met—NH₂ Molecular weight (Electrospray mass spectrum): m/Z, 1358 (M+H).

As would be apparent to skilled artisans, various changes and modifications are possible and are contemplated within the scope of the invention described. Although the compositions of the present invention are primarily directed at therapy, most of the compounds containing polycyclic aromatic chromophores can also be used for optical diagnostic imaging purposes.

What is claimed is:

1. An organic azide compound having the formula:
E—L—Ar—X—N₃
wherein Ar is an aromatic or a heteroaromatic radical derived from the group consisting of benzenes, polyfluorobenzenes, naphthalenes, naphthoquinones, anthracenes, anthraquinones, phenanthrenes, tetracenes, naphthacenediones, pyridines, quinolines, isoquinolines, indoles, isoindoles, pyrroles, imidiazoles, pyrazoles, pyrazines, purines, benzimidazoles, benzofurans, dibenzofurans, carbazoles, acridines, acridones, phenanthridines, thiophenes, benzothiophenes, dibenzothiophenes, xanthenes, xanthones, flavones, coumarins, and anthacylines;
E is selected from the group consisting of somatostatin receptor binding molecules, ST receptor binding molecules, neurotensin receptor binding molecules, bombesin receptor binding molecules, CCK receptor binding molecules, steroid receptor binding molecules, and carbohydrate receptor binding molecules;
L is selected from the group consisting of —(CH₂)ₐ—, —(CH₂)_{b}CONR¹—, —N(R²)CO(CH₂)_{c}—, —OCO(CH₂)_{d}—, —(CH₂)ₑCO₂—, —OCCNH—, —OCO₂—, —HNCONH—, —HNCSNH—, —HNNHCO—, —OSO₂—, —NR³(CH₂)ₑCONR⁴—, 13 CONR⁵(CH₂)_{f}NR⁶CO—, and —NR⁷CO(CH₂)_{g}CONR⁸—;
X is either a single bond or is selected from the group consisting of —(CH₂)ₕ—, —OCO—, —HNCO—, —(CH₂)ᵢCO—, and —(CH₂)ⱼOCO—;
R¹ to R⁸ are independently selected from the group consisting of hydrogen, C1-C10 alkyl, —OH, C1-C10 polyhydroxyalkyl, C1-C10 alkoxyl, C1-C10 alkoxyalkyl, —SO₃H, —(CH₂)ₖCO₂H, and —(CH₂)ₗNR⁹R¹⁰;
R⁹ and R¹⁰ are independently selected from the group consisting of hydrogen, C1-C10 alkyl, C5-C10 aryl, and C1-C10 polyhydroxyalkyl; and
subscripts a to I independently range from 0 to 10.

2. The compound of claim 1 wherein Ar is an aromatic or heteroaromatic radical derived from polyfluorbenzenes; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d—, —(CH}₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

3. The compound of claim 1 wherein Ar is an aromatic or heteroaromatic radical derived from anthraquinones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

4. The compound of claim 1 wherein Ar is an aromatic or heteroaromatic radical derived from napthacenediones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(C,H₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

5. The compound of claim 1 wherein Ar is an aromatic or heteroaromatic radical derived from indoles; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—,—HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁸R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

6. The compound of claim 1 wherein Ar is an aromatic or heteroaromatic radical derived from acridines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

7. The compound of claim 1 wherein Ar is an aromatic or heteroaromatic radical derived from acridones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCC—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

8. The compound of claim 1 wherein Ar is an aromatic or heteroaromatic radical derived from phenanthridines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)CO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

9. The compound of claim 1 wherein Ar is an aromatic or heteroaromatic radical derived from xanthones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢOCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

10. The compound of claim 1 wherein Ar is an aromatic or heteroaromatic radical derived from anthracyclines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑ—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

11. A method of performing a phototherapeutic procedure which comprises:
(a)administering an effective amount of an organic azide photosensitizer having the formula
E—L—Ar—X—N₃
wherein Ar is an aromatic or a heteroaromatic radical derived from the group consisting of benzenes, polyfluorobenzenes, naphthalenes, naphthoquinones, anthracenes, anthraquinones, phenanthrenes, tetracenes, naphthacenediones, pyridines, quinolines, isoquinolines, indoles, isoindoles, pyrroles, imidiazoles, pyrazoles, pyrazines, purines, benzimidazoles, benzofurans, dibenzofurans, carbazoles, acridines, acridones, phenanthridines, thiophenes, benzothiophenes, dibenzothiophenes, xanthenes, xanthones, flavones, coumarins, and anthacylines; E is a hydrogen atom or is selected from the group consisting of somatostatin receptor binding molecules, ST receptor binding molecules, neurotensin receptor binding molecules, bombesin receptor binding molecules, CCK receptor binding molecules, steroid receptor binding molecules, and carbohydrate receptor binding molecules; L is selected from the group consisting of —(CH₂)ₐ—, —(CH₂)_{b}CONR¹—, —N(R²)CO(CH₂)_{c}—, —OCO(CH₂)_{d}—, —(CH₂)ₑCO₂—, —OCONH—, —OCO₂—, —HNCONH—, —HNCSNH—, —HNNHCO—, —OSO₂—, —NR³(CH₂)ₑCONR⁴—, —CONR⁵(CH₂)_{f}NR⁶CO—, and —NR⁷CO(CH₂)_{g}CONR⁸—; X is either a single bond or is selected from the group consisting of —(CH₂)ₕ—, —OCO—, —HNCO—, —(CH₂)ᵢCO—, and —(CH₂)ⱼOCO—; R¹ to R⁸ are independently selected from the group consisting of hydrogen, C1-C10 alkyl, —OH, C1-C10 polyhydroxyalkyl, C1-C10 alkoxyl, C1-C10 alkoxyalkyl, —SO₃H, —(CH₂)ₖCO₂H, and —(CH₂)ₗNR⁹R¹⁰; R⁹ and R¹⁰ are independently selected from the group consisting of hydrogen, C1-C10 alkyl, C5-C10 aryl, and C1-C10 polyhydroxyalkyl; and subscripts a to I independently range from 0 to 10;
(b) allowing said photosensitizer to accumulate in target tissue; and
(c) exposing said target tissues with the light of wavelength between 300 and 950 nm with sufficient power and fluence rate to perform the phototherapeutic procedure.

12. The method of claim 11, wherein Ar is an aromatic or heteroaromatic radical derived from polyfluorbenzenes; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}13 , —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

13. The method of claim 11, wherein Ar is an aromatic or heteroaromatic radical derived from anthraquinones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

14. The method of claim 11, wherein Ar is an aromatic or heteroaromatic radical derived from napthacenediones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R¹⁰ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

15. The method of claim 11, wherein Ar is an aromatic or heteroaromatic radical derived from indoles; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂ )ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

16. The method of claim 11, wherein Ar is an aromatic or heteroaromatic radical derived from acridines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

17. The method of claim 11, wherein Ar is an aromatic or heteroaromatic radical derived from acridones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR¹—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

18. The method of claim 11, wherein Ar is an aromatic or heteroaromatic radical derived from phenanthridines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

19. The method of claim 11, wherein Ar is an aromatic or heteroaromatic radical derived from xanthones; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}—, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO —, and —(CH₂)ⱼOCO—, R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.

20. The method of claim 11, wherein Ar is an aromatic or heteroaromatic radical derived from anthracyclines; E is selected from the group consisting of somatostatin receptor binding molecules, ST receptor binding molecules, neurotensin receptor binding molecules, bombesin receptor binding molecules, CCK receptor binding molecules, and steroid receptor binding molecules; L is selected from the group consisting of —(CH₂)_{b}CONR¹—, —N(R²)CO(CH₂)_{c}—, —OCO(CH₂)_{d}, —(CH₂)ₑCO₂—, —HNCONH—, —HNCSNH—, and —NR⁷CO(CH₂)_{g}CONR⁸—; X is either a single bond or is selected from the group consisting of —(CH₂)ₕ—, —OCO—, —(CH₂)ᵢCO—, and —(CH₂)ⱼOCO—., R¹, R², R⁷ and R⁸ are independently selected from the group consisting of hydrogen, C1-C10 alkyl, C1-C10 polyhydroxyalkyl, —(CH₂)ₖCO₂H, and —(CH₂)ₗNR⁹R¹⁰; R⁹ and R¹⁰ are independently selected from the group consisting of hydrogen, C1-C10 alkyl, and C1-C10 polyhydroxyalkyl; and subscripts b-e and g-j independently range from 0 to 6.
